(19) United States
(12) Patent Application Publication
(10) Pub. No.: US 2024/0181123 A1
(43) Pub. Date: Jun. 6, 2024

(54) DRUG-ELUTING SUTURE FOR ATTENUATING INFLAMMATION IN WOUNDS

(71) Applicant: University of Utah Research Foundation, Salt Lake City, UT (US)

(72) Inventors: Azur AZAPAGIC, Salt Lake City, UT (US); Jayant P AGARWAL, Salt Lake City, UT (US); Himanshu J SANT, Salt Lake City, UT (US); Bruce K GALE, Salt Lake City, UT (US); Jill E SHEA, Salt Lake City, UT (US)

(21) Appl. No.: 18/527,086

(22) Filed: Dec. 1, 2023

Related U.S. Application Data
(60) Provisional application No. 63/429,390, filed on Dec. 1, 2022.

(51) Int. Cl. A61L 17/00; A61K 31/436; A61L 17/12

(52) U.S. Cl. CPC A61L 17/005; A61K 31/436; A61L 17/12; A61L 2300/41; A61L 2300/424; A61L 2300/426; B29C 48/92

(57) ABSTRACT

The present disclosure relates to a drug-eluting suture comprising an elongate strand formed of a polymer matrix. The suture includes an anti-inflammatory agent, such as tacrolimus, dispersed within the polymer matrix at a concentration ranging from 0.1% wt. to 5% wt., based on the total weight of the suture. The suture exhibits a porosity ranging from 1% to 20%. Under physiological conditions, the suture can demonstrate a mean anti-inflammatory agent release rate ranging from 0.1 ng/day to 100 ng/day for a period of at least 10 days.

### CROSS-REFERENCE TO RELATED APPLICATIONS

This application claims priority to and the benefit of U.S. Provisional Patent Application No. 63/429,390, filed Dec. 1, 2022 and titled DRUG-LOADED SUTURE MATERIALS, the entirety of which is incorporated herein by reference.

### BACKGROUND

Surgical sutures are widely used in wound closure and various other medical procedures, ranging from minor lacerations to complex surgical interventions. They are designed to hold tissues together until the wound has healed sufficiently to withstand normal stress.

One of the challenges in wound healing is the management of inflammation. Inflammation is a natural and integral part of the wound healing process. However, excessive or prolonged inflammation can lead to complications such as delayed healing, increased scarring, and even chronic wounds. Therefore, controlling inflammation is a major focus in wound management.

Despite the advancements in suture materials and wound management techniques, there is still a continuous search for improved methods and materials that can enhance wound healing, reduce inflammation, and minimize scarring.

### SUMMARY

This disclosure relates to a drug-eluting suture including a polymer matrix formed as an elongate strand and an anti-inflammatory agent dispersed within the polymer matrix. The polymer matrix can include a polycaprolactone (PCL)-based polymer such as poly-1-lactic acid-co-caprolactone (PLLA-PLC). The anti-inflammatory agent (e.g., tacrolimus) can be included at a concentration of 0.1% wt. to 5% wt., based on total weight of the suture, and the suture can have a porosity of 1% to 20%. The suture, under physiological conditions, exhibits a mean anti-inflammatory agent release rate of 0.1 ng/day to 100 ng/day for a period of at least 10 days.

Benefits of the disclosed drug-eluting sutures include: an optimal porosity that facilitates effective drug release and tissue integration while also maintaining effective mechanical properties: effective modulation of the inflammatory response at the treatment site and encouragement of an organized wound-healing process with reduced scar formation: effective maintenance of mechanical integrity and strength in healed tissues.

This disclosure also relates to a method of manufacturing a drug-eluting suture, the method including: mixing a polymer material within a first solvent composition to form a first mixture: mixing an anti-inflammatory agent within a second solvent composition to form a second mixture: combining the first and second mixtures to form a combined solution: and extruding the combined solution to form the drug-eluting suture, wherein the first solvent has a higher volatility than the second solvent, wherein the first solvent evaporates during extrusion to promote polymer precipitation, and wherein the anti-inflammatory agent is dispersed within a polymer matrix of the suture. The first solvent can include dichloromethane (DCM) and/or solvents with similar volatility. The second solvent can include dimethyl sulfoxide (DMSO) and/or solvents with similar solubility.

This disclosure also relates to a method of manufacturing a drug-eluting suture, the method including: mixing a polymer material with an anti-inflammatory agent, the anti-inflammatory agent including tacrolimus, to form a combined composition; raising the combined composition to a temperature above the glass transition temperature of the polymer material; and subjecting the combined composition to hot-melt extrusion to form the suture, wherein the temperature remains at or below 140° Celsius.

While many of the example applications disclosed herein relate to wound closure using the drug-eluting sutures, it will be understood that the sutures may be used in other applications where modulation of inflammation is desirable, including in repairs, connections, and/or treatments associated with other (non-dermal) tissues.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification.

FIG. 1 illustrates an example method of manufacturing a drug-eluting suture via a solvent extrusion and evaporation process.

FIGS. 2A and 2B illustrate scanning electron microscopy (SEM) images of a solvent-extruded “drug” PLLA-PCL suture incorporating tacrolimus (FIG. 2A) and a “placebo” PLLA-PCL suture that omits tacrolimus (FIG. 2B).

FIG. 2C compares the porosity of the drug PLLA-PCL suture and the placebo PLLA-PCL suture.

FIG. 3 illustrates the mean release rate of tacrolimus from the drug PLLA-PCL suture over time.

FIG. 4 illustrates the results of suture uniaxial tensile testing of the drug PLLA-PCL suture and the placebo PLLA-PCL suture.

FIG. 5 illustrates the results of uniaxial tensile strength testing of healed skin sutured with the drug PLLA-PCL suture and healed skin sutured with the placebo PLLA-PCL suture.

FIG. 6 illustrates histology results comparing mean epidermis thickness of mice tissue treated with the drug PLLA-PCL suture to mice tissue treated with a conventional poly(glycolide-co-caprolactone) (PGCL) suture.

FIG. 7 illustrates immunohistochemical analysis results comparing CD45 positive cells in basal and dermis layers of mice tissue treated with the drug PLLA-PCL suture to mice tissue treated with a conventional PGCL suture.

FIGS. 8A-8C illustrates histology results comparing collagen IV (FIG. 8A), integrin β4 (FIG. 8B), and keratinocytes (FIG. 8C) of mice tissue treated with a drug PLLA-PCL suture to tissue sections from a conventional PGCL suture treatment.

### DETAILED DESCRIPTION

### Overview

Anti-inflammatory agents, such as corticosteroids and non-steroidal anti-inflammatory drugs (NSAIDs), are commonly used to manage inflammation. These agents work by reducing the production of pro-inflammatory cytokines, which are proteins that regulate the immune response. However, these agents are typically administered systemically and can have side effects, especially when used for prolonged periods.

Another approach to managing inflammation is the use of immunosuppressive agents. One such agent is tacrolimus (also known as FK506), an immunosuppressant drug that is commonly used to prevent organ rejection in transplant patients. Tacrolimus works by inhibiting the activity of T cells, a type of white blood cell that plays a central role in the immune response. By suppressing T cell activity, tacrolimus can help to reduce inflammation and promote wound healing.

The present disclosure introduces a drug-eluting suture designed to enhance wound healing and reduce inflammation and scarring. The drug-eluting suture comprises a polymer matrix formed as an elongate strand and an anti-inflammatory agent dispersed within the polymer matrix. The anti-inflammatory agent can include tacrolimus.

### Polymer Matrix

In some embodiments, the polymer matrix can comprise a copolymer of lactic acid and caprolactone. This copolymer provides effective biodegradability and biocompatibility, making it an ideal material for use in sutures. For example, such polymers do not substantially swell when placed in a physiological environment such as a wound or incision site. This enables effective drug elution kinetics for lipophilic drugs to release based primarily on passive diffusion rather than being “flushed” out via water uptake into the polymer. Copolymers of lactic acid and caprolactone can also beneficially provide effective flexibility and mechanical strength, providing the sutures good resistance to breaking.

In some cases, the copolymer of lactic acid and caprolactone in the polymer matrix may comprise poly-1-lactic acid-co-caprolactone (PLLA-PLC). The PLLA-PLC may have a lactic acid to caprolactone ratio, on a molar percentage basis, of 30:70 to 90:10, such as 50:50 to 80:20, such as 70:30. This ratio can be adjusted to achieve desired properties in the suture, such as tensile strength, flexibility, and degradation rate.

The polymer matrix can additionally or alternatively include other polymers suitable for use in sutures. Examples include polyglycolic acid (PGA), polyglactin (PGLA), polylactic acid (PLA) polycaprolactone (PCL), polydioxanone (PDO), polyamide (nylon), polypropylene, polyester, and copolymers and/or combinations thereof.

The polymer matrix of the suture may have a porosity of 1% to 20%, such as 4% to 18%, such as 6% to 16%, such as 7% to 15%, such as 8% to 14%, or a range with any combination of the foregoing values as endpoints. The porosity of the polymer matrix can control the rate at which the anti-inflammatory agent is released from the suture and can also promote tissue integration. However, excessive porosity can also negatively affect mechanical properties of the suture such as tensile strength and ductility. For example, pores within the suture can act as stress concentrators, making the suture more susceptible to breakage under tension, or the pores can disrupt the continuity of the polymer chains, reducing the material's ability to deform or stretch without breaking. We have found that tuning the porosity of the suture to a level within the foregoing values beneficially provides an effective drug release profile without overly impacting desired mechanical properties of the suture.

Suture porosity can be determined using the following equation:

$${P}_{s} = \frac{{m}_{t} - {m}_{0}}{{V}_{s} \cdot ρ} 100$$

where Pₛ[%] is suture porosity, Vₛ[cm³] is the volume of the suture, mₜ[g] is the mass of the suture after incubation with ethanol for 24 hours, m₀[g] is the mass of the suture before incubation with ethanol, and ρ[g/cm³]=0.789 is the ethanol density.

### Anti-Inflammatory Agent

Dispersed within the polymer matrix is an anti-inflammatory agent. The concentration of the anti-inflammatory agent can be within the range of 0.1% wt. to 5% wt., such as 0.15% wt. to 2.5% wt., such as 0.2% wt. to 1.0% wt., or a range using any combination of the foregoing as endpoints, based on the total weight of the suture. This concentration can be adjusted to achieve desired therapeutic effects. The anti-inflammatory agent can comprise tacrolimus. For example, the suture can include tacrolimus at any of the concentration ranges recited above.

The amount of tacrolimus included in the suture can be tuned to balance the competing goals of scar tissue reduction and maintaining the strength of a healing wound. Effective healing requires a certain level of tissue inflammation. Excessive inflammation can lead to overgrowth of scar tissue: however, insufficient inflammation may compromise the strength of the sutured wound. We have found that including tacrolimus within the amounts listed above beneficially reduces excess scar tissue without significantly affecting the strength of the resulting healed wound.

In some cases, the anti-inflammatory agent may additionally or alternatively more other anti-inflammatory and/or immunosuppressant agents. The additional anti-inflammatory agents may comprise a macrolactam (e.g., rapamycin, pimecrolimus, cyclosporine, ascomycin, tacrolimus analogs), corticosteroid, and/or non-steroidal anti-inflammatory drug. These agents can provide additional mechanisms of action to further reduce inflammation and/or promote wound healing. In embodiments that include tacrolimus and one or more additional agents, the additional agents can work synergistically with tacrolimus to provide a more comprehensive anti-inflammatory and/or immunosuppressant effect.

The suture is designed to release the anti-inflammatory agent in a controlled and sustained manner under physiological conditions. For example, the suture can be configured to release the anti-inflammatory agent (e.g., tacrolimus) at a mean rate of 0.1 ng/day to 100 ng/day, such as 1 ng/day to 50 ng/day, such as 4 ng/day to 20 ng/day, such as 7 ng/day to 15 ng/day, or a range with any of the foregoing as endpoints, over a sustained period of at least 10 days, at least 15 days, at least 20 days, at least 21 days, or even longer depending on particular application needs. The controlled and sustained release of the anti-inflammatory agent (e.g., tacrolimus) beneficially modulates the inflammatory response at the wound site, thereby promoting wound healing and reducing scarring.

The suture may exhibit consistent drug release with minimal variability over time, which can promote effective healing as compared to a drug release profile that too quickly washes out or that lacks sufficient consistency for effective anti-inflammatory activity. For example, the suture can be configured to provide a mean anti-inflammatory agent release rate that varies by no more than 10 ng/day over a period of at least 10 days, or at least 15 days, or at least 20 days, or at least 21 days, or even longer according to particular application needs, when exposed to physiological conditions.

The anti-inflammatory agent release rate can be determined by immersing a drug-eluting suture in an Eppendorf tube or other suitable container with an appropriate buffer (e.g., 1×phosphate-buffered saline (PBS)), collecting the media and any released tacrolimus at scheduled time points (e.g., daily, every 2-5 days, or other suitable sampling schedule), and replacing with fresh media. The collected media samples can be subsequently analyzed using enzyme-linked immunoassay (ELISA) and/or other appropriate analytical technique(s) to quantify the amount of tacrolimus within each media sample. For each media sample, dividing the determined amount of tacrolimus by the number of days the media sample was in contact with the suture will provide the mean release rate as that term is used herein.

### Suture Mechanical Properties

In some embodiments, the suture may have a diameter of 50 μm to 400 μm, such as 100 μm to 300 μm, though other suture sizes can also be utilized depending on application needs. For example, any size corresponding to suture sizes as defined by the United States Pharmacopeia (USP) may be utilized (e.g., size 11-0 to size 7). A smaller diameter suture may be used for delicate or superficial wounds or ophthalmic applications, while a larger diameter suture may be more suitable for deep or large wounds or orthopedic applications.

The tensile strength of the suture provides the ability to withstand the forces exerted on it during wound closure and healing. In some cases, the suture may exhibit a uniaxial tensile strength of at least 0.8 N (e.g., for a suture with 170 μm diameter). This level of tensile strength enables the suture to withstand the tension exerted on it during wound closure without breaking, thereby maintaining the integrity of the wound closure and promoting effective healing.

In some embodiments, the polymer matrix may be subjected to an annealing process, which involves heating the suture to a specific temperature and then slowly cooling it. This process can enhance the mechanical properties of the polymer matrix, such as its tensile strength and ductility. Following the annealing process, the suture may exhibit a uniaxial tensile strength increase of up to 25%. For example, the suture may exhibit a uniaxial tensile strength of at least 1.0 N (e.g., for a suture with 170 μm diameter). This increased tensile strength further enhances the suture's ability to withstand tension during wound closure and healing, thereby ensuring the integrity and effectiveness of the wound closure.

### Anti-Adherent Agent

In some cases, the suture may further comprise an anti-adherent agent. The anti-adherent agent may comprise magnesium stearate. The anti-adherent agent aids in effective manufacture of the suture. Polymer powders (used to form the polymer matrix) often tend to clump together with the anti-inflammatory agent. For example, PCL-based polymer powders tend to form clumps with tacrolimus. This disrupts flow during the extrusion process and can also result in sub-par products, such as sutures that do not obtain the preferred porosity and/or drug-loading properties disclosed herein.

The magnesium stearate may be included at a concentration of 0.1% wt. to 2.5% wt., such as 0.5% wt. to 2.0% wt, such as 1.0% wt. to 1.5% wt., or a range using any combination of the foregoing as endpoints, based on the total weight of the suture.

The anti-adherent agent can additionally or alternatively include other anti-adherent compounds known in the art, such as stearic acid, other salts of stearic acid, talc, silicon dioxide, polyethylene glycol, cellulose (e.g., microcrystalline cellulose (MCC)), carnauba wax, and/or salts of stearyl fumarate (e.g., sodium stearyl fumarate).

### Example Methods of Manufacture

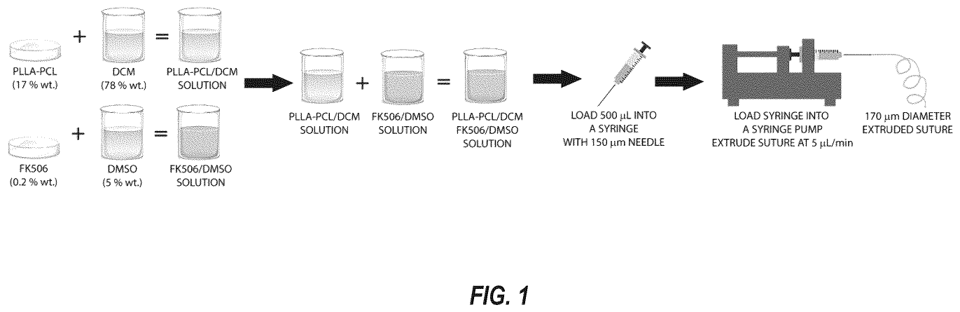

An example method of manufacturing the drug-eluting suture includes mixing a polymer material within a first solvent composition to form a first mixture, mixing an anti-inflammatory agent within a second solvent composition to form a second mixture, combining the first and second mixtures to form a combined solution, and extruding the combined solution to form the drug-eluting suture. An example of such a method is illustrated in FIG. 1.

In some cases, the first solvent used in the manufacturing process may comprise dichloromethane (DCM), and the second solvent may comprise dimethyl sulfoxide (DMSO). DCM has a relatively high volatility, which allows it to evaporate during the extrusion process, promoting polymer precipitation and the formation of the suture. Solvents that can be used in addition to or as an alternative to DCM include, for example, solvents with similar boiling points to DCM such as acetone, ethyl acetate, methyl ethyl ketone (MEK), tetrahydrofuran (THF), and/or diethyl ether. Solvents that can be used in addition to or as an alternative to DMSO include, for example, solvents with relatively high solubility such as dimethylformamide (DMF), dimethylacetamide (DMAc), acetonitrile, and/or ethanol.

The suture can optionally be annealed. We discovered that annealing a suture manufactured using the solvent evaporation process can beneficially increase the tensile strength of the suture, in some cases by up to 20-25%. The annealing process can include heating the formed drug-eluting suture to a temperature of 90° C. to 100° C., such as 95° C., for a period of 6 hours or more, or 12 hours or more, such as 24 hours to 120 hours, such as 72 hours.

In another variation of the manufacturing process involving hot-melt extrusion, a mixed/combined composition of the polymer material (often in the form of a powder) and the anti-inflammatory agent is raised to a temperature above the glass transition temperature of the polymer material. The combined composition is then subjected to hot-melt extrusion to form the suture. The temperature during the hot-melt extrusion process preferably remains at or below 140° C. We discovered that tacrolimus begins to degrade at temperatures above this limit. By carrying out the hot-melt extrusion at temperatures at or below 140° C., the process allows for the formation of the suture without causing degradation of the tacrolimus or the polymer material.

### Additional Terms & Definitions

As used herein, the term “physiological environment” describes the conditions a suture is exposed to when used on a typical subject, such as when used to close a wound and/or incision in soft tissue such as dermal tissue. For example, physiological pH is typically about 6 to 8, though dermal tissue is often mildly acidic (e.g., 4.7 to 5.8). Physiological temperatures are typically about 36° to 38° C., and fluids in a physiological environment typically have a tonicity that is isotonic (e.g., equivalent to about 0.9% w/v saline solution).

While certain embodiments of the present disclosure have been described in detail, with reference to specific configurations, parameters, components, elements, etcetera, the descriptions are illustrative and are not to be construed as limiting the scope of the claimed invention.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition.

At the very least, and not as an attempt to limit the application of the doctrine of equivalents to the scope of the claims, each numerical parameter can be construed in light of the number of reported significant digits and by applying ordinary rounding techniques. However, values given in one context with a particular number of significant digits should not be interpreted as limiting the scope of values in other instances of this disclosure.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “a suture”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be omitted or essentially omitted from the disclosed embodiments. For example, polymer components or anti-inflammatory agents not specifically recited herein may be completely omitted or essentially omitted.

An embodiment that “essentially omits” or is “essentially free of” a component may include trace amounts and/or non-functional amounts of the component. For example, an “essentially omitted” component may be included in an amount no more than 1%, no more than 0.1%, no more than 0.01%, or no more than 0.001% by total weight of the composition.

A composition that “completely omits” or is “completely free of” a component does not include a detectable amount of the component (i.e., does not include an amount above any inherent background signal associated with the testing instrument) when analyzed using standard analysis techniques such as, for example, chromatographic techniques (e.g., thin-layer chromatography (TLC), gas chromatography (GC), liquid chromatography (LC)), or spectroscopy techniques (e.g., Fourier transform infrared (FTIR) spectroscopy).

Furthermore, it should be understood that for any given element of component of a described embodiment, any of the possible alternatives listed for that element or component may generally be used individually or in combination with one another, unless implicitly or explicitly stated otherwise.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

### EXAMPLES

### Example 1: Drug-Eluting Suture Fabrication Process

As shown in FIG. 1, the drug suture fabrication process involved room-temperature solvent extrusion and evaporation using a syringe pump and a needle. FK506 (5.8 mg) was dissolved in dimethyl sulfoxide (DMSO) (800 μL), and PLLA-PCL (2.882 g) was dissolved in dichloromethane (DCM) (10 mL) under ambient conditions. The DMSO/FK506 and DCM/PLLA-PCL solutions were combined and subjected to mechanical mixing. This composite solution was loaded into a glass syringe (500 μL, Hamilton Gastight #1750) with an extrusion needle (150 μm diameter, McMaster-Carr #75165A129) and inserted into a syringe pump. The mixture was extruded (5 μL/min) to produce the drug PLLA-PCL suture (13.8 mg, 170 μm diameter, 0.2% wt. FK506).

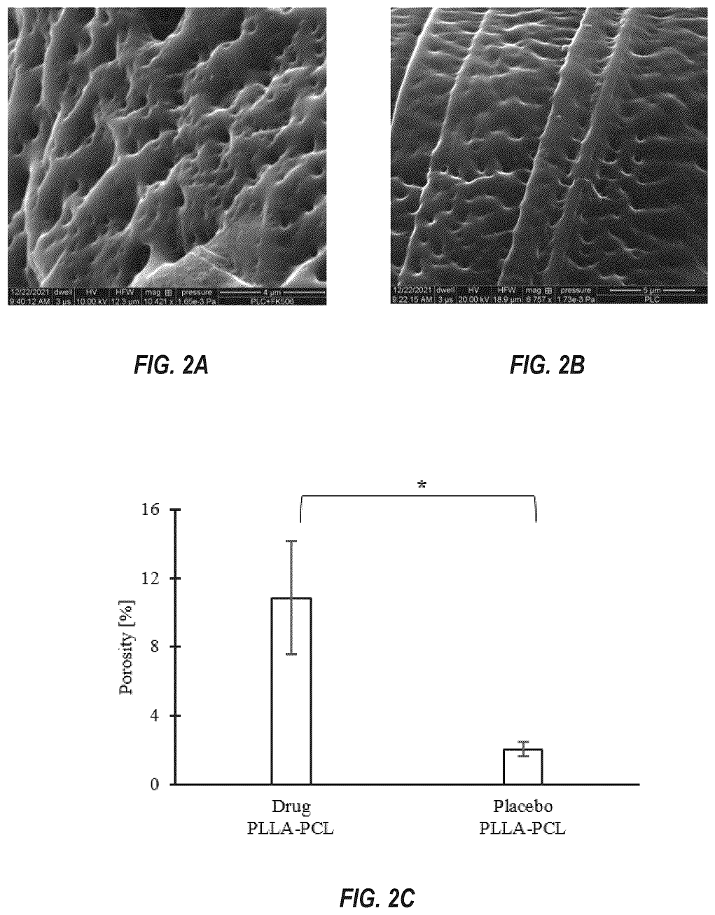

The drug PLLA-PCL suture (13.8 mg, 170 μm diameter, 0.2% wt. FK506) was on average about 9% more porous compared to a placebo PLLA-PCL suture (13.8 mg, 170 μm diameter, 0.0% wt. FK506) suture control group (n=3 per group). The formation of a porous structure in solvent-extruded sutures resulted most likely from the balance between solvent evaporation, polymer precipitation, and extrusion dynamics. FIGS. 2A and 2B illustrate SEM images of the drug PLLA-PCL suture (FIG. 2A) and the placebo PLLA-PCL suture (FIG. 2B), and FIG. 2C compares the porosity of the drug PLLA-PCL suture and the placebo PLLA-PCL suture.

### Example 2: Drug Release Testing

To replicate in-vivo conditions, we conducted tests at 37° C. The procedure entailed immersing the drug PLLA-PCL suture in Eppendorf tubes containing 1×phosphate-buffered saline (PBS) (1 mL) (n=3). We collected PBS+released FK506 media (1 mL) at 5, 8, 11, 16, and 21 days and replaced it with fresh PBS (1 mL). Subsequent analysis of the collected media, using enzyme-linked immunoassay (ELISA), quantified the FK506 release rates. At the end of the 21-day test, the daily mean release rates were recorded and reported with 95% confidence intervals.

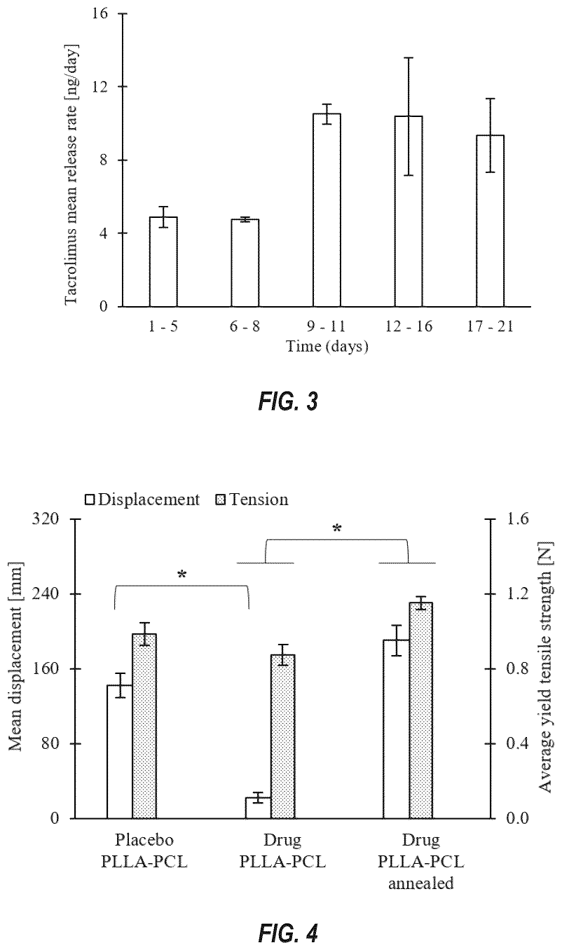

Results are shown in FIG. 3. As shown, release rates from the PLLA-PCL suture averaged about 10 ng/day. This illustrates the suture's potential for sustained and consistent drug delivery.

### Example 3: Suture Uniaxial Strength Testing

The mechanical strength of the suture was evaluated by uniaxial tensile strength testing using the Instron testing system (Norwood, MA, USA). Tests were conducted on drug PLLA-PCL sutures (13.8 mg, 170 μm diameter, 0.2% wt. FK506) and placebo PLLA-PCL sutures (13.8 mg, 170 μm diameter, 0.0% wt. FK506) as a control group (n=3 per group). This comparative approach investigated the effect of solvent extrusion and FK506 on the suture peak tensile strength and the displacement at failure. Each test specimen was secured within the Instron system's clamping mechanism and subjected to a tensile displacement (1 mm/s) until failure. The mean peak tensile strength and the displacement at the failure were recorded and reported with 95% confidence intervals.

Results are shown in FIG. 4. The mean peak tensile strength of PLLA-PCL was 99 MPa (n=3). Assuming the linear elasticity, the anticipated theoretical suture tensile peak strength was 2.25 N. However, during our observations, both test groups achieved around 45% of the theoretical strength. The difference in the mean tensile strength between the drug PLLA-PCL suture and placebo PLLA-PCL suture groups was not statistically significant (p>0.05). However, the difference in the mean displacement at failure between the drug PLLA-PCL suture and placebo PLLA-PCL suture groups was statistically significant (p≤ 0.05). We annealed the drug PLLA-PCL suture at 95° C. for 72 h and retested its tensile strength. The annealing increased the drug PLLA-PCL suture tensile strength, reaching approximately 60% of the theoretical value, improved ductility, and the displacement at failure. The difference in the mean peak tensile strength and displacement at failure between the annealed and not annealed drug PLLA-PCL suture groups was statistically significant (p≤0.05).

### Example 4: Mouse Epidermal Wound Closure

We conducted in-vivo wound closure experiments on mice using the drug PLLA-PCL suture (13.8 mg, 170 μm diameter, 0.2% wt. FK506) and placebo poly glycolic-co-caprolactone (PGCL) Ethicon suture as a control group (United States Pharmacopeia (USP) 5-0) (male, Mus musculus, 10-12 weeks, 30 grams mean, n=4 per group). We used a baseball stitch technique, consisting of 7-8 stitches and five knots, to close an incision (30 mm) on the back of the mice. We took photographs weekly and, in week five, harvested the mice. During the necropsy, we excised a 30 W×40 L mm section of the healed skin containing the sutured incision. Half of each sample was used for mechanical strength testing reported in this Example.

The other half underwent pathological and immunofluorescence staining to study morphological changes in the healed tissue (Examples 5-7). We preserved the skin samples in a 4% paraformaldehyde solution for a few hours, then rinsed and stored them in a glycine-PBS solution before embedding. The samples were then prepared for microscopic examination. They were dehydrated using a graded ethanol series, cleared in Bioclear, and embedded in paraffin. We cut sections (4 μm thick), placed them on slides, and dried them overnight.

The mechanical integrity of healed skin is important in evaluating the efficacy of wound treatments. One of the primary concerns when attenuating inflammation is the potential compromise in the mechanical strength of the resultant tissue. Over-attenuation of inflammation could lead to incomplete or improper matrix formation, resulting in healed skin lacking its undamaged counterpart's robustness. Ideally, tissue heals in a manner that limits inflammation but does not sacrifice mechanical properties of the treated tissue. The mechanical uniaxial strength of the healed skin from the wound closure experiments on mice was tested using the Instron testing system (Norwood, MA, USA). Each test specimen was secured within the Instron system's clamping mechanism and subjected to a tensile displacement (1 mm/s) normal to the incision until failure. The mean peak force at failure was recorded and reported with 95% confidence intervals.

From week one to week five, there was a noticeable difference in the appearance of wounds sutured with the drug PLLA-PCL suture compared to those with the placebo PGCL Ethicon suture. The observed trend was consistent across all animals in the study, with similar outcomes noted for the entire cohort. The wounds closed with the drug PLLA-PCL suture displayed less redness around the incision area. By the end of the study at week five, the wounds closed with the drug PLLA-PCL suture displayed less noticeable redness than the placebo PGCL Ethicon suture control group.

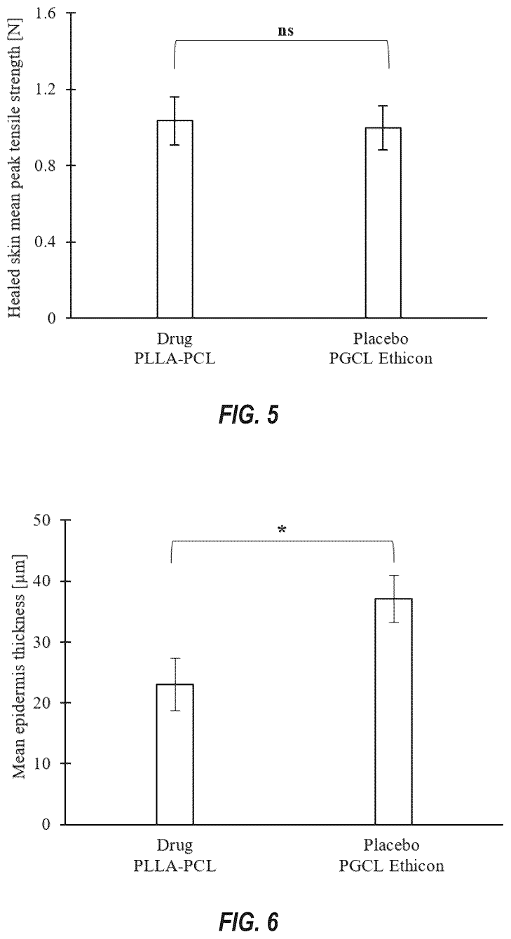

As shown in FIG. 5, the mechanical strength of the healed wound was not adversely affected by the presence of FK506. When comparing the drug PLLA-PCL suture with the placebo PGCL Ethicon suture control group, there was no statistically significant difference in the healed skin mean tensile strength (p>0.05). This suggests that the FK506 release rates did not compromise the mechanical integrity of the healed skin and did not overly attenuate inflammation. A balanced inflammatory response is essential for optimal wound healing, and excessive suppression could hinder the process. The results suggest that the drug PLLA-PCL suture strikes an appropriate balance, allowing for effective wound healing without compromising the mechanical properties of the healed tissue.

### Example 5: Histological Analysis of Skin Tissue

Thickening of the epidermis is characteristic of scar tissue, accompanied by dense collagen fibers, in contrast to the thinner and less dense structure observed in healthy skin. For Hematoxylin and Eosin (H&E) staining, slides were immersed in hematoxylin (0.1%, Ricca #3530-32) for ten minutes, rinsed in water, treated with eosin (0.1%, Cancer Diagnostics Inc. #EM00PG) for five minutes, and then rinsed again. They were dehydrated with graded ethanol and mounted using Distrene 80 Plasticizer Xylene (DPX) (Fluka). For the MTS staining using Masson Trichrome with Aniline Blue kit (Bio-Optica), the procedure began by applying a mixture of each of Weigert's iron hematoxylin solutions A (six drops) and B (six drops) to the sections, allowing them to act for ten minutes. Without a rinse, the slides were drained and treated with an alcoholic picric acid solution (ten drops) for four minutes. A brief rinse in distilled water was followed by adding Ponceau acid fuchsin (ten drops) for four minutes. After another rinse, phosphomolybdic acid solution (ten drops) was added for ten minutes. Without rinsing, the slides were drained and treated with aniline blue (ten drops) for five minutes. They were then dehydrated, cleared in Bioclear (Bio-Optica), and mounted in DPX (Fluka). Post-staining slides were digitized using the ZEISS Axioscan at 20× magnification. Epidermis thickness was measured using ZEN 3.1 software. The mean epidermis thickness was recorded and reported with 95% confidence intervals.

In the drug PLLA-PCL suture, the scar tissue exhibited a lower collagen density and a thinner epidermal layer, as shown in FIG. 6. These findings illustrate that the drug PLLA-PCL suture scar tissue more closely emulates the characteristics of healthy skin, indicating its potential to attenuate scarring and improve wound healing outcomes.

### Example 6: Immunohistochemical Analysis of Skin Tissue

CD45, a leukocyte marker, is a crucial indicator of the inflammatory response. Reducing CD45-positive cells in tissues indicates decreased inflammation. The immunohistochemical (IHC) process began with epitope retrieval, heating tissue sections with Bond Epitope Retrieval Solution to expose the antigens. This WAS followed by blocking non-specific protein interactions and incubating the cells with a rabbit polyclonal anti-CD45 antibody. Using the bond refine detection kit, the staining process highlights the antibody-antigen complexes for clear visualization. Post-staining, the slides underwent a dehydration process through a series of ethanol baths, followed by clearing in xylene. The final analysis of the IHC images was conducted using QuPath 0.4.3 software on the ZEISS Axioscan multispectral imaging platform. The StarDist nucleus segmentation algorithm aids in cell detection at 20× magnification. Nuclear outlines were expanded to delineate cell borders, and DAPI staining was employed to classify cells into positive and negative categories. A consistent DAPI threshold across all samples ensures uniformity in the analysis. The percentage of positive cells was then calculated.

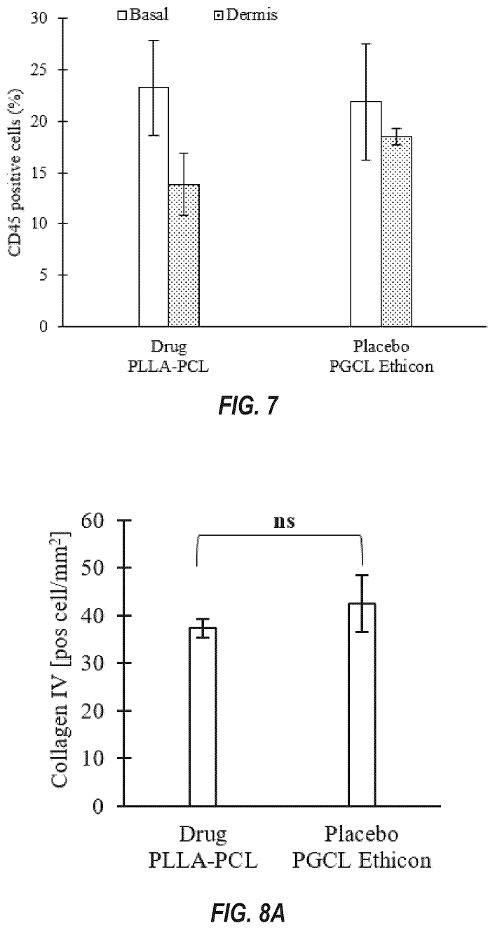

As shown in FIG. 7, the drug PLLA-PCL group showed a lower mean value in the dermis layer than the placebo PGCL Ethicon group, indicating a trend towards the drug PLLA-PCL sutures being more effective in reducing inflammation and scar tissue in the dermis.

### Example 7: Immunofluorescence Analysis of Skin Tissue

Integrin B4 is typically abundant in the basal layer of healthy skin, but is reduced in scar tissue. Elevated levels of pro-inflammatory cytokines can suppress integrin β4 expression, undermining the skin's structural integrity and delaying wound healing.

A multiplex immunofluorescence (mIF) panel detection process employed tyramide-conjugated fluorophores from PerkinElmer (Catalog No. NEL801001KT) and was executed on the Leica Bond Rx autostainer. Formalin-fixed paraffin-embedded (FFPE) tissue sections (4 μm thickness) underwent deparaffinization and sequential hydration. After antigen retrieval in ER1 pH6 (AR9961-Bond™ Epitope Retrieval 1) or ER2 pH9 (AR9640-Bond™ Epitope Retrieval 2) for 30 minutes at 95° C., primary antibodies were incubated for 30 minutes. This was followed by the secondary antibody (anti-rabbit HRP polymer) and the Opal TSA fluorophore application, each for ten minutes (520-FP1487001KT, 620-FP1495001KT, and 690-FP1497001KT). Heat-mediated stripping was done between each antibody staining round. After staining all target markers, tissues were counterstained with spectral 4′,6-diamidino-2-phenylindole (DAPI) for five minutes, rinsed, and mounted using ProLong Diamond Antifade Mountant (Life Technology, Catalog No. P36961, Carlsbad, CA). Table 1 shows antibody application dilution ratios and fluorophore pairing.

TABLE 1
Multiplex immunofluorescence (mIF) staining.

| Antibodies | CD104 | Collagen IV | AE1/AE3 |
|---|---|---|---|
| Dilutions | 1:200 | 1:1000 | 1:500 |
| Maker | Thermofisher | Abcam | Novus |
| Clone | JM11-06 | EPR22911 | Polyclonal |
| Retrieval | ER2 for 30 mi | ER2 for 30 mi | ER1 for 30 mi |
| Opal Fluorophores | 520 | 620 | 690 |
| Opal Dilutions | 1:100 | 1:100 | 1:100 |

We used the ZEISS Axioscan multispectral imaging platform to scan the fluorescence slide at 20× magnification between 420 and 690 nm. mIF images were analyzed with QuPath 0.4.3 software. A pixel-classifier identified the basal area, and the dermis area was determined by subtracting the basal area from the tissue area. The StarDist nucleus segmentation algorithm detected cells at 20× magnification. Cell borders were expanded by five pixels from nuclear outlines. Marker thresholds were set and applied to each sample for phenotype counting, with positive cell counts normalized to the respective area (mm²).

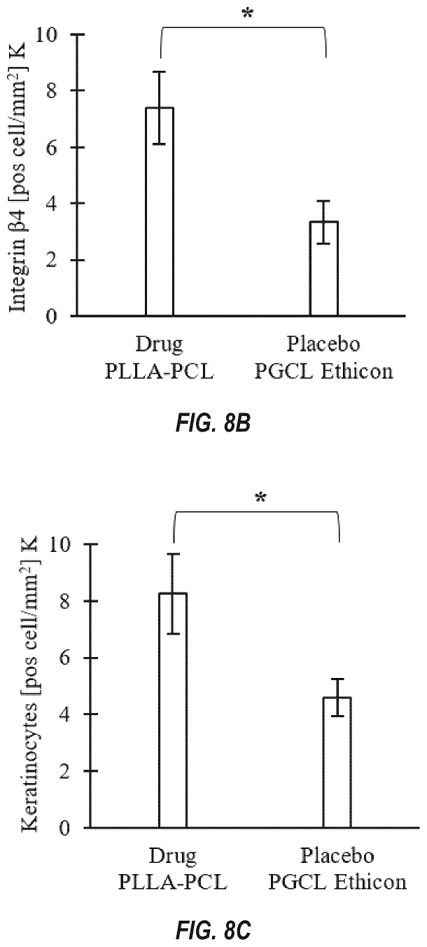

Both suture types demonstrated similar levels of collagen IV expression, suggesting a comparable structural foundation in the extracellular matrix (FIG. 8A). However, differences were observed in the expression of integrin β4 (FIG. 8B) and the presence of keratinocytes (FIG. 8C), where the drug PLLA-PCL suture group exhibited upregulation of integrin β4 expression and an increased presence of keratinocytes. These features support effective wound healing and restoration of the skin's barrier function.

1. A drug-eluting suture, comprising:
a polymer matrix formed as an elongate strand; and
an anti-inflammatory agent dispersed within the polymer matrix, wherein the anti-inflammatory agent is included at a concentration of 0.1% wt. to 5% wt., based on total weight of the suture,
wherein the suture has a porosity of 1% to 20%, and
wherein the suture, under physiological conditions, exhibits a mean anti-inflammatory agent release rate of 0.1 ng/day to 100 ng/day for a period of at least 10 days.

2. The suture of claim 1, wherein the anti-inflammatory agent comprises tacrolimus and wherein the tacrolimus is included at a concentration of 0.1% wt. to 5% wt., based on total weight of the suture.

3. The suture of claim 1, wherein the polymer matrix comprises poly-1-lactic acid-co-caprolactone (PLLA-PLC).

4. The suture of claim 3, wherein the PLLA-PLC has a lactic acid to caprolactone ratio, on a molar percentage basis, of 50:50 to 80:20.

5. The suture of claim 1, wherein the suture exhibits a mean anti-inflammatory agent release rate of 4 ng/day to 20 ng/day for a period of at least 21 days.

6. The suture of claim 1, wherein the suture exhibits a mean anti-inflammatory agent release rate that varies by no more than 10 ng/day over a period of at least 10 days when exposed to physiological conditions.

7. The suture of claim 1, wherein the anti-inflammatory agent comprises tacrolimus and further comprises one or more additional immunosuppressant and/or anti-inflammatory agents.

8. The suture of claim 7, wherein the one or more additional immunosuppressant and/or anti-inflammatory agents comprise a macrolactam, corticosteroid, and/or non-steroidal anti-inflammatory drug.

9. The suture of claim 1, wherein the suture has a diameter of 50 μm to 400 μm.

10. The suture of claim 1, wherein the suture exhibits a uniaxial tensile strength of at least 0.8 N.

11. The suture of claim 1, wherein the polymer matrix is annealed and wherein the suture exhibits a uniaxial tensile strength of at least 1.0 N.

12. The suture of claim 1, further comprising an anti-adherent agent.

13. The suture of claim 12, wherein the anti-adherent agent comprises magnesium stearate, wherein the magnesium stearate is included at a concentration of 0.1% wt. to 2.5% wt., based on total weight of the suture.

14. The suture of claim 1, wherein the suture has a porosity of 4% to 18%.

15. A method of manufacturing a drug-eluting suture, the method comprising:
mixing a polymer material within a first solvent composition to form a first mixture;
mixing an anti-inflammatory agent within a second solvent composition to form a second mixture;
combining the first and second mixtures to form a combined solution; and
extruding the combined solution to form the drug-eluting suture,
wherein the first solvent has a higher volatility than the second solvent,
wherein the first solvent evaporates during extrusion to promote polymer precipitation, and
wherein the anti-inflammatory agent is dispersed within a polymer matrix of the suture.

16. The method of claim 15, wherein the first solvent comprises dichloromethane (DCM) and/or the second solvent comprises dimethyl sulfoxide (DMSO).

17. The method of claim 15, wherein the polymer material includes a copolymer of lactic acid and caprolactone, and/or wherein the anti-inflammatory agent comprises tacrolimus.

18. The method of claim 15, wherein the polymer matrix has a porosity of 1% to 20%.

19. A method of manufacturing a drug-eluting suture, the method comprising:
mixing a polymer material with an anti-inflammatory agent, the anti-inflammatory agent comprising tacrolimus, to form a combined composition;
raising the combined composition to a temperature above the glass transition temperature of the polymer material; and
subjecting the combined composition to hot-melt extrusion to form the suture,
wherein the temperature remains at or below 140° C.

20. The method of claim 19, wherein the polymer material comprises a copolymer of lactic acid and caprolactone.
